SEQUENCE LISTING (12 sequences)

SEQ ID NO: 1 - DNA, 377 bp, synthetic construct
  source 1..377: mol_type = other DNA
actcgcgcgcacggagcgacgacacccccgcgcgtgcacccgctcgggacaggagccggactcctgtgcagcttccctcggccgccgggggcctccccgcgcctcgccggcctccaggccccctcctggctggcgagcgggcgccacatctggcccgcacatctgcgctgccggcccggcgcggggtccggagagggcgcggcgcggaggcgcagccaggggtccgggaaggcgccgtccgctgcgctgggggctcggtctatgacgagcagcggggtctgccatgggtcgggggctgctcaggggcctgtggccgctgcacatcgtcctgtggacgcgtatcgccagcacgatcccaccgcacgttcagaagtcgg

SEQ ID NO: 2 - DNA, 800 bp, synthetic construct
  source 1..800: mol_type = other DNA
aatataacaccagcaatcctgacttgttgctagtcatatttcaagtgacaggcatcagcctcctgccaccactgggagttgccatatctgtcatcatcatcttctactgctaccgcgttaaccggcagcagaagctgagttcaacctgggaaaccggcaagacgcggaagctcatggagttcagcgagcactgtgccatcatcctggaagatgaccgctctgacatcagctccacgtgtgccaacaacatcaaccacaacacagagctgctgcccattgagctggacaccctggtggggaaaggtcgctttgctgaggtctataaggccaagctgaagcagaacacttcagagcagtttgagacagtggcagtcaagatctttccctatgaggagtatgcctcttggaagacagagaaggacatcttctcagacatcaatctgaagcatgagaacatactccagttcctgacggctgaggagcggaagacggagttggggaaacaatactggctgatcaccgccttccacgccaagggcaacctacaggagtacctgacgcggcatgtcatcagctgggaggacctgcgcaagctgggcagctccctcgcccgggggattgctcacctccacagtgatcacactccatgtgggaggcccaagatgcccatcgtgcacagggacctcaagagctccaatatcctcgtgaagaacgacctaacctgctgcctgtgtgactttgggctttccctgcgtctggaccctactctgtctgtggatgacctggctaacagtgggcag

SEQ ID NO: 3 - DNA, 23 bp, synthetic construct
  source 1..23: mol_type = other DNA
  misc_feature 21..23: note = PAM site
  misc_feature 1..20: note = TGFBR2 gRNA target sequence
tgctggcgatacgcgtccacagg

SEQ ID NO: 4 - DNA, 23 bp, synthetic construct
  source 1..23: mol_type = other DNA
  misc_feature 1..20: note = TGFBR2 gRNA target sequence
  misc_feature 21..23: note = PAM site
tcggtctatgacgagcagcgggg

SEQ ID NO: 5 - DNA, 23 bp, synthetic construct
  source 1..23: mol_type = other DNA
  misc_feature 1..20: note = TGFBR2 gRNA target sequence
  misc_feature 21..23: note = PAM site
aacgtgcggtgggatcgtgctgg

SEQ ID NO: 6 - DNA, 23 bp, synthetic construct
  source 1..23: mol_type = other DNA
  misc_feature 1..20: note = TGFBR2 gRNA target sequence
  misc_feature 21..23: note = PAM site
ggacgatgtgcagcggccacagg

SEQ ID NO: 7 - DNA, 23 bp, synthetic construct
  source 1..23: mol_type = other DNA
  misc_feature 1..20: note = TGFBR2 gRNA target sequence
  misc_feature 21..23: note = PAM site
ctcggtctatgacgagcagcggg

SEQ ID NO: 8 - DNA, 23 bp, synthetic construct
  source 1..23: mol_type = other DNA
  misc_feature 1..20: note = TGFBR2 gRNA target sequence
  misc_feature 21..23: note = PAM site
caagaggcatactcctcataggg

SEQ ID NO: 9 - DNA, 23 bp, synthetic construct
  source 1..23: mol_type = other DNA
  misc_feature 1..20: note = TGFBR2 gRNA target sequence
  misc_feature 21..23: note = PAM site
ccacgccaagggcaacctacagg

SEQ ID NO: 10 - DNA, 23 bp, synthetic construct
  source 1..23: mol_type = other DNA
  misc_feature 1..20: note = TGFBR2 gRNA target sequence
  misc_feature 21..23: note = PAM site
ccaagatgcccatcgtgcacagg

SEQ ID NO: 11 - DNA, 23 bp, synthetic construct
  source 1..23: mol_type = other DNA
  misc_feature 1..20: note = TGFBR2 gRNA target sequence
  misc_feature 21..23: note = PAM site
aaagcgacctttccccaccaggg

SEQ ID NO: 12 - DNA, 23 bp, synthetic construct
  source 1..23: mol_type = other DNA
  misc_feature 1..20: note = TGFBR2 gRNA target sequence
  misc_feature 21..23: note = PAM site
gccgcgtcaggtactcctgtagg

(19) United States
(12) Patent Application Publication
(10) Pub. No.: US 2023/0372485 A1
(43) Pub. Date: Nov. 23, 2023

(54) ENGINEERED T CELLS WITH REDUCED TGF-BETA RECEPTOR SIGNALING

(71) Applicant: Neogene Therapeutics B.V., Amsterdam (NL)

(72) Inventors: Vanessa M. TUBB, Amsterdam (NL); Jeroen W J VAN HEIJST, Amsterdam (NL); Gavin M. BENDLE, Amsterdam (NL)

(21) Appl. No.: 18/055,370

(22) Filed: Nov. 14, 2022

Related U.S. Application Data
(60) Provisional application No. 63/279,551, filed on Nov. 15, 2021.
(60) Provisional application No. 63/337,091, filed on Apr. 30, 2022.

(51) Int. Cl. A61K 39/00; C12N 9/22; C12N 15/11; C12N 15/90; C07K 14/71

(52) U.S. Cl. CPC A61K 39/464401; C12N 9/22; C12N 15/111; C12N 15/907; C07K 14/71; A61K 39/4611; A61K 39/4631; A61K 39/4632; C12N 2310/20

(57) ABSTRACT

T cells comprising an engineered genomic modification of the TGFBR2 gene are provided. The genomic modification can reduce receptor surface expression and/or reduce TGF-β induced signaling, and allows T cells having such TGFBR2 disruption to continue to proliferate and continue to kill target tumor cells even in the presence of physiologically relevant levels of TGF-β. In preferred embodiments, the T cells are further engineered to express a CAR or exogenous TCR. Methods of making the engineered T cells, pharmaceutical compositions comprising populations of such T cells, and methods of treating are also provided.

### 1. CROSS REFERENCE TO RELATED APPLICATIONS

This application claims the benefit of and priority to U.S. Provisional Application No. 63/279,551, filed on Nov. 15, 2021, and 63/337,091, filed on Apr. 30, 2022, which are incorporated herein by reference in their entireties for all purposes.

### 2. SEQUENCE LISTING

The instant application contains a Sequence Listing which has been submitted via Patent Center and is hereby incorporated by reference in its entirety. Said XML copy, created on Nov. 14, 2022, is named 50557US_sequencelisting.xml, and is 20,432 bytes in size.

### 3. BACKGROUND OF THE INVENTION

Human T cells that have been redirected to recognize antigens expressed on tumor cells, either through expression of a chimeric antigen receptor (“CAR”) or expression of an exogenous T cell receptor (TCR), have proven to be effective in treating certain hematologic cancers. So far, however, CAR-T and TCR-T approaches have shown poorer efficacy against solid tumors, in part due to the presence of an immunosuppressive tumor microenvironment. Improved CAR-T and TCR-T cell approaches that are capable of overcoming the suppressive tumor microenvironment of solid cancers are needed.

### 4. SUMMARY OF THE INVENTION

Transforming growth factor beta (“TGF-β”) is an immunosuppressive cytokine found within the tumor microenvironment of some solid cancers, such as advanced metastatic solid cancers. In some circumstances, TGF-β may limit anti-tumor immune responses. One possible mechanism for the effect of TGF-β is the suppression of T cell functionality, including T cell cytotoxicity, proliferation, and cytokine production. Without limiting the present invention, it is currently believed that TGF-β binds to the extracellular domain of transforming growth factor beta receptor 2 (“TGFBR2”), promoting dimerization of TGFBR2 with TGFBR1. Following receptor dimerization, the TGFBR2 kinase domain transphosphorylates TGFBR1, resulting in downstream phosphorylation of SMAD2 and SMAD3 and subsequent expression of TGF-β responsive genes.

We have now discovered that targeted disruption of the TGFBR2 gene can reduce receptor surface expression and/or reduce TGF-β induced signaling, and allows TCR-T cells having such TGFBR2 disruption to continue to proliferate and continue to kill target tumor cells even in the presence of physiologically relevant levels of TGF-β.

According, in a first aspect, this disclosure provides T cells that comprise an engineered genomic modification of the TGFBR2 gene, wherein the engineered genomic modification results in a level of surface-expressed TGFBR2, or a detectable portion thereof, that is between about 20% and about 60% of the level of surface-expressed TGFBR2 on a matched control cell. In some embodiments, the genomic modification is in exon 4 of the TGFBR2 gene. In some embodiments, the T cell expresses an exogenous TCR or a CAR, preferably an exogenous TCR.

In another aspect, T cells comprising an engineered genomic modification of the TGFBR2 gene are presented, wherein the modification results in a surface-expressed TGFBR2 that is truncated. In some embodiments, the genomic modification is in exon 4 of the TGFBR2 gene. In some embodiments, the T cell expresses an exogenous TCR or a CAR, preferably an exogenous TCR. In some embodiments, the TCR or CAR is integrated into a defined place in the genome of the T cell. In certain embodiments, the integration is performed using CRISPR, optionally CRISPR-Cas9.

In another aspect, methods of making such engineered T cells are provided. In another aspect, pharmaceutical compositions comprising such engineered T cells are provided. In a further aspect, methods of treating a patient are provided, the methods comprising administering to a patient a therapeutically effective amount of the engineered T cells or pharmaceutical compositions comprising such engineered T cells.

### 5. BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 provides a schematic depicting the exon structure of the human TGF-β receptor 2 gene (“TGFBR2”). The human TGFBR2 gene comprises 7 exons, encoding—from N terminus to C terminus—an extracellular domain, a transmembrane domain, and a kinase domain. The binding sites for guide RNAs (“gRNA”) that were used to target an RNA-guided nuclease respectively to exon 1 (TGFBR2 gRNA1-gRNA7) and exon 4 (TGFBR2 gRNA15-gRNA22) are shown. The gRNA target sequences are presented in Table 2.

FIGS. 2A and 2B present data showing that RNA-guided nuclease editing that disrupts the TGFBR2 gene renders T cells resistant to TGF-β-induced phospho Smad2/3 upregulation. Healthy donor human T cells were engineered to express the 1G4 TCR and then were electroporated with various TGFBR2 gRNA RNP complexes. 1G4 TCR-expressing TGFBR2-edited T cells were then cultured in the presence or absence of TGF-β (20 ng/mL) in duplicates for 30 minutes, and then intracellularly stained with an antibody detecting phosphorylated Smad2/3 protein. FIG. 2A presents flow cytometry histograms depicting phospho Smad2/3 expression following treatment with (gray) or without (black) TGF-β. Peak size is normalized to the modal value for each curve. FIG. 2B summarizes fold change in phospho Smad2/3 median fluorescence intensity (“MFI”) upon TGF-β treatment. Error bars represent standard deviation of duplicates.

FIG. 3 presents data showing that disrupting the TGFBR2 gene in T cells by RNA-guided nuclease editing results in reduced TGFBR2 surface expression. Healthy donor human T cells were engineered to express the 1G4 TCR and then electroporated with various clinical grade TGFBR2 gRNA RNP complexes targeting either the extracellular domain (“ECD”) (gRNAs 1 and 4-7) or intracellular kinase domain (“ICD”) (gRNAs 15-17, 20 and 22) of TGFBR2. Four days after editing, TGFBR2 surface expression was analysed by surface staining with an anti-TGFBR2 antibody. TGFBR2 expression is normalized to TGFBR2 median fluorescence intensity in T cells engineered to express 1G4 TCR without TGFBR2 gene editing. Error bars represent standard deviation of duplicates.

FIGS. 4A.1-4D present data showing that T cells with a nuclease-mediated disruption in the TGFBR2 gene have superior cytotoxic function following repetitive tumor cell challenge in the presence of TGF-β. 1G4 TCR-expressing T cells (control) and 1G4 TCR-expressing T cells with various edited disruptions to the TGFBR2 gene were subjected to a repetitive cytotoxicity assay using the IncuCyte platform. T cells were cultured with A375-GFP⁺ target cells at an effector-to-target ratio of 5:1 in the presence or absence of exogenous TGF-β (20 ng/mL) for about 72 hours, before being harvested and re-cultured with fresh A375-GFP⁺ cells for a total of 4 rounds. A375-GFP⁺ cell killing was imaged using a 10× objective every 2 hours and quantified by counting the remaining GFP⁺ cells in cultures. FIG. 4A.1 shows A375-GFP⁺ cell counts when A375-GFP⁺ target cells were cultured for one and two rounds in the presence of T cells lacking exogenous 1G4 TCR and without any edits to the TGFBR2 gene (“non-edited”), 1G4-expressing T cells lacking edits to the TGFBR2 gene (“1G4 TCR only”), and no T cells (“No T cells”), with and without TGF-β. FIG. 4A.2 shows A375-GFP⁺ cell counts when A375-GFP⁺ target cells were cultured for three and four rounds, in the presence of T cells lacking exogenous 1G4 TCR and without any edits to the TGFBR2 gene (“non-edited”), 1G4-expressing T cells lacking edits to the TGFBR2 gene (“1G4 TCR only”), and no T cells (“No T cells”), with and without TGF-β. FIG. 4B.1 shows A375-GFP⁺ cell counts when A375-GFP⁺ target cells were cultured for one and two rounds in the presence of T cells expressing exogenous 1G4 TCR and in which the TGFBR2 gene was disrupted using exon 1-targeting gRNAs (TGFBR2-1 to TGFBR2-7), with and without TGF-β. FIG. 4B.2 shows A375-GFP⁺ cell counts when A375-GFP⁺ target cells were cultured for three and four rounds in the presence of T cells expressing exogenous 1G4 TCR and in which the TGFBR2 gene was disrupted using exon 1-targeting gRNAs (TGFBR2-1 to TGFBR2-7), with and without TGF-β. FIG. 4C.1 shows A375-GFP⁺ cell counts when A375-GFP⁺ target cells were cultured for one and two rounds in the presence of T cells expressing exogenous 1G4 TCR and in which the TGFBR2 gene was disrupted using exon 4-targeting gRNAs (TGFBR2-15, TGFBR2-16, TGFBR2-17, TGFBR2-20, TGFBR2-22), with and without TGF-β. Error bars represent the standard deviation of triplicates. FIG. 4C.2 shows A375-GFP⁺ cell counts when A375-GFP⁺ target cells were cultured for three and four rounds in the presence of T cells expressing exogenous 1G4 TCR and in which the TGFBR2 gene was disrupted using exon 4-targeting gRNAs (TGFBR2-15, TGFBR2-16, TGFBR2-17, TGFBR2-20, TGFBR2-22), with and without TGF-β. Error bars represent the standard deviation of triplicates. FIG. 4D is a histogram compiling the A375-GFP⁺ cell counts after 4 rounds of challenge. Error bars represent the standard deviation of triplicates.

FIG. 5 presents data demonstrating that T cells having a nuclease-mediated disruption in the TGFBR2 gene maintain their proliferative capacity in the presence of TGF-β. TGFBR2-disrupted, 1G4 TCR-expressing T cells were subjected to repetitive restimulation using ImmunoCult (10 μL/mL) in the presence or absence of exogenous TGF-β (20 ng/mL). T cell proliferation was quantified by counting T cells on a weekly basis. Error bars represent the standard deviation of duplicates.

FIGS. 6A-6B presents data demonstrating that a nuclease-mediated disruption of the TGFBR2 gene does not alter expression of an exogenous TCR. Cells were stained with antibodies detecting the endogenous TCR (“huTCR”) or the exogenous TCR marked with the mur6 epitope (“mur6”). FIG. 6A presents FACS plots showing that expression of the knocked-in TCR was similar among three different disruption sites 7 days after electroporation and 14 days after electroporation and selection of cells containing the TCR repair template. FIG. 6B presents FACS plots showing that expression of the knocked-in TCR was similar among two different disruption sites 7 days after electroporation and 14 days after electroporation and selection of cells containing the TCR repair template.

### 6. DETAILED DESCRIPTION OF THE INVENTION

### 6.1. Definitions

A “matched control cell” is one that is as closely identical to a modified cell as scientifically acceptable and practicable in order to conduct a scientifically valid comparison. Other than the genomic modification that is the subject of the comparison, an appropriate control cell should have the same cell type, same growth conditions, and/or same other modifications. Persons of skill in the art will understand what variables, parameters, and conditions are relevant in any given context in order to determine any differences (e.g., changes in levels of surface expression) resulting from a genomic modification. The contents of this application provide examples of appropriate control cells in certain contexts.

### 6.2. T Cells Comprising an Engineered Genomic Modification of the TGFBR2 Gene

In a first aspect, T cells comprising an engineered genomic modification of the TGFBR2 gene are provided. The engineered genomic modification results in a level of surface-expressed TGFBR2, or a detectable portion thereof, that is between about 20% and about 60% of the level of surface-expressed TGFBR2 on a matched control cell.

In some embodiments, the engineered genomic modification results in a level of surface-expressed TGFBR2, or detectable portion thereof, that is about 20%, 25%, 30%, 35%, 40%, 45%, 50%, 55% or 60% of the level of surface-expressed TGFBR2 on a matched control cell. In some embodiments, the engineered genomic modification results in a level of surface-expressed TGFBR2, or detectable portion thereof, that is no more than about 20%, 25%, 30%, 35%, 40%, 45%, 50%, 55% or 60% of the level of surface-expressed TGFBR2 on a matched control cell.

In some embodiments, the T cell is a CD8+ αβ T cell, a CD4+αβ T cell, or a γδ T cell. In some embodiments, the T cell is a human T cell. In some human T cell embodiments, the T cell is obtained from a cancer patient. In some human T cell embodiments the T cell is obtained from a healthy subject. In some human T cell embodiments, the T cell is a progeny cell of a T cell obtained from a cancer patient or obtained from a healthy subject.

In some embodiments, the surface-expressed TGFBR2, or detectable portion thereof, is capable of binding TGF-β but not phosphorylating TGFBR1. In some embodiments, the T cell cannot effectively signal through Smad2/3 in response to contact of the T cell with physiologically relevant levels of TGF-β.

In various embodiments, the engineered genomic modification comprises one or more of (i) an insertion and/or a deletion in the TGFBR2 gene promoter, (ii) a frame-shifting insertion and/or deletion in an exon of the TGFBR2 gene, (iii) a deletion of a part, but not the entirety, of the coding region of the TGFBR2 gene, (iv) a substitution, insertion, and/or deletion that creates a stop codon in an exon upstream of the native stop codon, and (v) a substitution, insertion, and/or deletion that modifies one or more donor and/or acceptor RNA splice sites within the TGFBR2 gene.

In certain embodiments, the genomic modification is in exon 1 of the TGFBR2 gene, exon 2 of the TGFBR2 gene, exon 3 of the TGFBR2 gene, exon 4 of the TGFBR2 gene, exon 5 of the TGFBR2 gene, exon 6 of the TGFBR2 gene, or exon 7 of the TGFBR2 gene. In particular embodiments, the genomic modification is in exon 4 of the TGFBR2 gene.

In various embodiments, the genomic modification is effected by an RNA-guided nuclease. In some embodiments, the RNA-guided nuclease is a double strand break-inducing nuclease. In some embodiments, the RNA-guided nuclease is a single strand break-inducing nuclease (nickase). In some embodiments, the RNA-guided nuclease is fused to a second enzyme. In particular embodiments, the second enzyme is a reverse transcriptase.

In specific embodiments, the genomic modification is a frameshift caused by an RNA-guided nuclease cut between bases 294 and 295, 389 and 390, 543 and 544, 547 and 548, or 674 and 675 of exon 4 of the TGFBR2 gene (SEQ ID NO: 2).

In some embodiments, the T cell expresses an exogenous TCR or a CAR. In certain embodiments, the TCR is introduced into the T cell using viral methods. In certain embodiments, the TCR is introduced into the T cell using methods (e.g., CRISPR-Cas9 or other CRISPR enzymes) that integrate a gene for expressing the exogenous TCR into a specific site in the genome of the T cell.

In certain preferred embodiments, the T cell expresses an exogenous TCR. In particular embodiments, the T cell continues to express its endogenous TCR. In particular embodiments, the T cell does not express its endogenous TCR.

In certain embodiments, the T cell expresses a CAR. In particular embodiments, the CAR is a first generation CAR. In some embodiments, the CAR is a second generation CAR. In some embodiments, the CAR is a parallel CAR as described in U.S. Pat. No. 10,703,794, the disclosure of which is incorporated herein by reference in its entirety. In some embodiments, the CAR is an NKG2D-based CAR as described in WO 2021/058563, the disclosure of which is incorporated herein by reference in its entirety.

In some embodiments, the exogenous TCR or CAR recognizes a tumor antigen. As is understood by the person of skill in the art, the “antigen” recognized by a TCR is a peptide-HLA complex (pHLA). In some embodiments, the tumor antigen is a tumor-associated antigen that is also expressed by non-tumor cells. In some embodiments, the tumor antigen is a cancer/testis antigen. In some embodiments, the tumor antigen is a neoantigen. In certain embodiments, the neoantigen is a shared, or public, tumor neoantigen. In certain embodiments, the neoantigen is a non-shared, or private, neoantigen.

In some embodiments, the T cell maintains the ability to kill a population of target cells that express the antigen recognized by the exogenous TCR or CAR in vitro in the presence of physiologically relevant levels of TGF-β after at least two exposure events to the target cells. In some embodiments, the T cell maintains the ability to kill a population of target cells that express the antigen recognized by the exogenous TCR or CAR in vitro for at least about 72, 80, 90, 100, 110, 120, 130, 140, 144, 150, 160, 170, 180, 190, 200, 210, 220, 230, 240, 250, 260, 270, 280, or 288 hours in the presence of physiologically relevant levels of TGF-β.

In another aspect, T cells comprising an engineered genomic modification of the TGFBR2 gene are provided. The modification results in a surface-expressed TGFBR2 that is truncated.

In some embodiments, the T cell is a CD8+ αβ T cell, a CD4+ αβ T cell, or a γδ T cell. In some embodiments, the T cell is a human T cell. In some human T cell embodiments, the T cell is obtained from a cancer patient. In some human T cell embodiments, the T cell is obtained from a healthy subject. In some human T cell embodiments, the T cell is a progeny cell of a T cell obtained from a cancer patient or obtained from a healthy subject.

In some embodiments, the surface-expressed truncated TGFBR2 is capable of binding TGF-β but not phosphorylating TGFBR1. In some embodiments, the T cell cannot effectively signal through Smad2/3 in response to contact of the T cell with physiologically relevant levels of TGF-β.

In some embodiments, the engineered genomic modification comprises one or more of (i) a frame-shifting insertion and/or deletion in exon 4 of the TGFBR2 gene, (ii) a deletion of exons 5 to 7 of the TGFBR2 gene, optionally with a full or partial deletion of exon 4, and (iii) a substitution, insertion, and/or deletion in exon 4 that creates a premature stop codon.

In certain embodiments, the genomic modification is in exon 1 of the TGFBR2 gene, exon 2 of the TGFBR2 gene, exon 3 of the TGFBR2 gene, exon 4 of the TGFBR2 gene, exon 5 of the TGFBR2 gene, exon 6 of the TGFBR2 gene, or exon 7 of the TGFBR2 gene. In particular embodiments, the genomic modification is in exon 4 of the TGFBR2 gene.

In various embodiments, the genomic modification is effected by an RNA-guided nuclease. In some embodiments, the RNA-guided nuclease is a double strand break-inducing nuclease. In some embodiments, the RNA-guided nuclease is a single strand break-inducing nuclease (nickase). In some embodiments, the RNA-guided nuclease is fused to a second enzyme. In particular embodiments, the second enzyme is a reverse transcriptase.

In specific embodiments, the genomic modification is a frameshift caused by an RNA-guided nuclease cut between bases 294 and 295, 389 and 390, 543 and 544, 547 and 548, or 674 and 675 of exon 4 of the TGFBR2 gene (SEQ ID NO: 2).

In some embodiments, the T cell expresses an exogenous TCR or a CAR. In certain embodiments, the TCR is introduced into the T cell using viral methods. In certain embodiments, the TCR is introduced into the T cell using methods (e.g., CRISPR-Cas9 or other CRISPR enzymes) that integrate a gene for expressing the exogenous TCR into a specific site in the genome of the T cell.

In certain preferred embodiments, the T cell expresses an exogenous TCR. In particular embodiments, the T cell continues to express its endogenous TCR. In particular embodiments, the T cell does not express its endogenous TCR.

In certain embodiments, the T cell expresses a CAR. In particular embodiments, the CAR is a first generation CAR. In some embodiments, the CAR is a second generation CAR. In some embodiments, the CAR is a parallel CAR as described in U.S. Pat. No. 10,703,794, the disclosure of which is incorporated herein by reference in its entirety. In some embodiments, the CAR is an NKG2D-based CAR as described in WO 2021/058563, the disclosure of which is incorporated herein by reference in its entirety.

In some embodiments, the T cell expresses an exogenous TCR or CAR that recognizes a tumor antigen. As is understood by the person of skill in the art, the “antigen” recognized by a TCR is a peptide-HLA complex (pHLA). In some embodiments, the tumor antigen is a tumor-associated antigen that is also expressed by non-tumor cells. In some embodiments, the tumor antigen is a cancer/testis antigen. In some embodiments, the tumor antigen is a neoantigen. In certain embodiments, the neoantigen is a shared, or public, tumor neoantigen. In certain embodiments, the neoantigen is a non-shared, or private, neoantigen.

In some embodiments, the T cell maintains the ability to kill a population of target cells that express the antigen recognized by the exogenous TCR or CAR in vitro in the presence of physiologically relevant levels of TGF-β after at least two exposure events to the target cells. In some embodiments, the T cell maintains the ability to kill a population of target cells that express the antigen recognized by the exogenous TCR or CAR in vitro for at least about 72, 80, 90, 100, 110, 120, 130, 140, 144, 150, 160, 170, 180, 190, 200, 210, 220, 230, 240, 250, 260, 270, 280, or 288 hours in the presence of physiologically relevant levels of TGF-β.

In some embodiments, the T cell has an enhanced cytokine response after contact or exposure to a tumor or other cell line presenting an antigen recognized by a TCR expressed by the T cell. In certain embodiments, the cytokine is interferon-γ, interleukin-2, or tumor necrosis factor-a.

### 6.3. Pharmaceutical Compositions

In another aspect, pharmaceutical compositions are provided that comprise a T cell as described herein and a pharmaceutically acceptable carrier. In preferred embodiments, the T cells express an exogenous TCR or CAR.

In various embodiments, the pharmaceutical composition comprises a population of T cells as described herein. In certain embodiments, the T cells express an exogenous TCR or CAR. In particular embodiments, all of the T cells in the population express the same exogenous TCR. In particular embodiments, all of the T cells in the population express the same CAR. In particular embodiments, the pharmaceutical composition comprises T cells as described herein, wherein the T cells in the population collectively express a plurality of CARs.

In some embodiments, the pharmaceutical composition is adapted for administration by intravenous infusion. In some embodiments, the composition is adapted for intratumoral administration.

### 6.4. Methods of Engineering T Cells

In another aspect, methods are provided for making the TGFBR2-modified T cells described herein. In some embodiments, the methods comprise modifying the TGFBR2 gene in the T cell genome, wherein following gene modification, the level of surface-expressed TGFBR2 or a detectable portion thereof is between about 20% and about 60% of the level of surface-expressed TGFBR2 on a matched control cell.

In some embodiments, the engineered genomic modification results in a level of surface-expressed TGFBR2, or detectable portion thereof, that is about 20%, 25%, 30%, 35%, 40%, 45%, 50%, 55% or 60% of the level of surface-expressed TGFBR2 on a matched control cell. In some embodiments, the engineered genomic modification results in a level of surface-expressed TGFBR2, or detectable portion thereof, that is no more than about 20%, 25%, 30%, 35%, 40%, 45%, 50%, 55% or 60% of the level of surface-expressed TGFBR2 on a matched control cell.

In some embodiments, the T cell is a CD8+ αβ T cell, a CD4+ αβ T cell, or a γδ T cell. In some embodiments, the T cell is a human T cell. In some human T cell embodiments, the T cell is obtained from a cancer patient. In some human T cell embodiments the T cell is obtained from a healthy subject. In some human T cell embodiments, the T cell is a progeny cell of a T cell obtained from a cancer patient or obtained from a healthy subject.

In some embodiments, the surface-expressed TGFBR2, or detectable portion thereof, is capable of binding TGF-β but not phosphorylating TGFBR1. In some embodiments, the T cell cannot effectively signal through Smad2/3 in response to contact of the T cell with physiologically relevant levels of TGF-β.

In some embodiments, the modification is one or more of (i) an insertion and/or a deletion in the TGFBR2 gene promoter, (ii) a frame-shifting insertion and/or deletion in an exon of the TGF-βIIR gene, (iii) a deletion of a part, but not the entirety, of the coding region of the TGFBR2 gene, (iv) a substitution, insertion, and/or deletion that creates a stop codon in an exon upstream of the native stop codon, and (v) a substitution, insertion, and/or deletion that modifies one or more donor and/or acceptor RNA splice sites within the TGFBR2 gene. In particular embodiments, the modification is in exon 4 of the TGFBR2 gene.

In some embodiments, modifying comprises introducing an RNA-guided nuclease and at least one RNA guide into the T cell. In some embodiments, the RNA-guided nuclease is a double strand break-inducing nuclease. In some embodiments, the RNA-guided nuclease is a single strand break-inducing nuclease (nickase). In some embodiments, the RNA-guided nuclease is fused to a second enzyme. In particular embodiments, the second enzyme is a reverse transcriptase.

In some embodiments, the RNA-guided nuclease cuts between bases 294 and 295, 389 and 390, 543 and 544, 547 and 548, or 674 and 675 of exon 4 of the TGFBR2 gene (SEQ ID NO: 2). In certain embodiments, the at least one guide RNA has the sequence of SEQ ID NOs:8-12.

In some embodiments, the method further comprises a subsequent step of selecting a T cell having the desired genomic modification.

In some embodiments, the method further comprises the step, before or after modifying the TGFBR2 gene, of engineering the T cell to express a CAR or an exogenous TCR. In certain embodiments, the T cell is engineered to express an exogenous TCR. In certain embodiments, the TCR is introduced into the T cell using viral methods. In certain embodiments, the TCR is introduced into the T cell using methods (e.g., CRISPR-Cas9 or other CRISPR enzymes) that integrate a gene for expressing the exogenous TCR into a specific site in the genome of the T cell. In some embodiments, the T cell concurrently expresses its endogenous TCR. In some embodiments, the T cell has been further engineered so as to not express its endogenous TCR.

In some embodiments, the exogenous TCR or CAR recognizes a tumor antigen. In some embodiments, the tumor antigen is a tumor-associated antigen that is also expressed by non-tumor cells. In some embodiments, the tumor antigen is a cancer/testis antigen. In some embodiments, the tumor antigen is a neoantigen. In certain embodiments, the neoantigen is a shared, or public, tumor neoantigen. In certain embodiments, the neoantigen is a non-shared, or private, neoantigen.

In various embodiments, following modification of the TGFBR2 gene and further engineering the T cell to express a CAR or exogenous TCR, the T cell maintains the ability to kill a population of target cells that express the antigen recognized by the exogenous TCR or CAR in vitro in the presence of physiologically relevant levels of TGF-β after at least two exposure events to the target cells. In some embodiments, the T cell maintains the ability to kill a population of target cells that express the antigen recognized by the exogenous TCR or CAR in vitro for at least about 72, 80, 90, 100, 110, 120, 130, 140, 144, 150, 160, 170, 180, 190, 200, 210, 220, 230, 240, 250, 260, 270, 280, or 288 hours in the presence of physiologically relevant levels of TGF-β.

In another aspect, methods are provided for making the TGFBR2-modified T cells described herein, wherein the modification is within exon 4 and results in a surface-expressed TGFBR2 that is truncated.

In some embodiments, the T cell is a CD8+ αβ T cell, a CD4+ αβ T cell, or a γδ T cell. In some embodiments, the T cell is a human T cell. In some human T cell embodiments, the T cell is obtained from a cancer patient. In some human T cell embodiments the T cell is obtained from a healthy subject. In some human T cell embodiments, the T cell is a progeny cell of a T cell obtained from a cancer patient or obtained from a healthy subject.

In some embodiments, the surface-expressed truncated TGFBR2 is capable of binding TGF-β but not phosphorylating TGFBR1. In some embodiments, the T cell cannot effectively signal through Smad2/3 in response to contact of the T cell with physiologically relevant levels of TGF-β.

In some embodiments, the truncated, surface-expressed, TGFBR2 is present at levels equal to, or greater than, the amount of TGFBR2 present in an unmodified T cell. In some embodiments, the truncated, surface-expressed, TGFBR2 is present at levels between about 20% and 60% of the level of TGFBR2 present in an unmodified T cell.

In some embodiments, the engineered genomic modification comprises one or more of (i) a frame-shifting insertion and/or deletion in exon 4 of the TGFBR2 gene, (ii) a deletion of exons 5 to 7 of the TGFBR2 gene, optionally with a full or partial deletion of exon 4, and (iii) a substitution, insertion, and/or deletion in exon 4 that creates a premature stop codon.

In some embodiments, modifying comprises introducing an RNA-guided nuclease and at least one RNA guide into the T cell. In some embodiments, the RNA-guided nuclease is a double strand break-inducing nuclease. In some embodiments, the RNA-guided nuclease is a single strand break-inducing nuclease (nickase). In some embodiments, the RNA-guided nuclease is fused to a second enzyme. In particular embodiments, the second enzyme is a reverse transcriptase.

In some embodiments, the RNA-guided nuclease cuts between bases 294 and 295, 389 and 390, 543 and 544, 547 and 548, or 674 and 675 of exon 4 of the TGFBR2 gene (SEQ ID NO: 2). In certain embodiments, the at least one guide RNA has the sequence of SEQ ID NOs:8-12.

In some embodiments, the method further comprises a subsequent step of selecting a T cell having the desired genomic modification.

In some embodiments, the method further comprises the step, before or after modifying the TGFBR2 gene, of engineering the T cell to express a CAR or an exogenous TCR. In certain embodiments, the T cell is engineered to express an exogenous TCR. In certain embodiments, the TCR is introduced into the T cell using viral methods. In certain embodiments, the TCR is introduced into the T cell using methods (e.g., CRISPR-Cas9 or other CRISPR enzymes) that integrate a gene for expressing the exogenous TCR into a specific site in the genome of the T cell. In some embodiments, the T cell concurrently expresses its endogenous TCR. In some embodiments, the T cell has been further engineered so as to not express its endogenous TCR.

In some embodiments, the exogenous TCR or CAR recognizes a tumor antigen. In some embodiments, the tumor antigen is a tumor-associated antigen that is also expressed by non-tumor cells. In some embodiments, the tumor antigen is a cancer/testis antigen. In some embodiments, the tumor antigen is a neoantigen. In certain embodiments, the neoantigen is a shared, or public, tumor neoantigen. In certain embodiments, the neoantigen is a non-shared, or private, neoantigen.

In various embodiments, following modification of the TGFBR2 gene and further engineering the T cell to express a CAR or exogenous TCR, the T cell maintains the ability to kill a population of target cells that express the antigen recognized by the exogenous TCR or CAR in vitro in the presence of physiologically relevant levels of TGF-β after at least two exposure events to the target cells. In some embodiments, the T cell maintains the ability to kill a population of target cells that express the antigen recognized by the exogenous TCR or CAR in vitro for at least about 72, 80, 90, 100, 110, 120, 130, 140, 144, 150, 160, 170, 180, 190, 200, 210, 220, 230, 240, 250, 260, 270, 280, or 288 hours in the presence of physiologically relevant levels of TGF-β.

In another aspect, engineered T cells produced by the methods described herein are provided.

### 6.5. Methods of Treating Disease

In another aspect, methods are provided for treating a subject in need of treatment. In typical embodiments, the subject is a human patient. The method comprises administering to the subject, typically a human patient, a therapeutically effective amount of engineered T cells as described herein or pharmaceutical compositions comprising such engineered T cells, as described herein.

In some embodiments, the engineered T cells are engineered from T cells obtained from, or the progeny of T cells obtained from, the patient to be treated (autologous treatment). In some embodiments, the engineered T cells are engineered from T cells obtained from, or the progeny of T cells obtained from, one or more individuals other than the patient to be treated (allogeneic treatment).

In some embodiments, the engineered T cells are administered by intravenous infusion. In some embodiments, the engineered T cells are administered by intratumoral administration.

In some embodiments, the engineered T cells have an enhanced cytokine response after contact or exposure to a tumor or other cell line presenting an antigen recognized by a TCR expressed by the T cell. In certain embodiments, the cytokine is interferon-γ or tumor necrosis factor-α.

### 6.6. EXAMPLES

### 6.6.1. Example 1: TGFBR2-Disrupted, TCR-Expressing T Cells are Resistant to TGF-β Signaling

This example shows that disrupting the TGFBR2 gene in T cells that are further engineered to express an exogenous TCR reduces surface expression of TGFBR2 and renders the T cells resistant to TGF-β signaling.

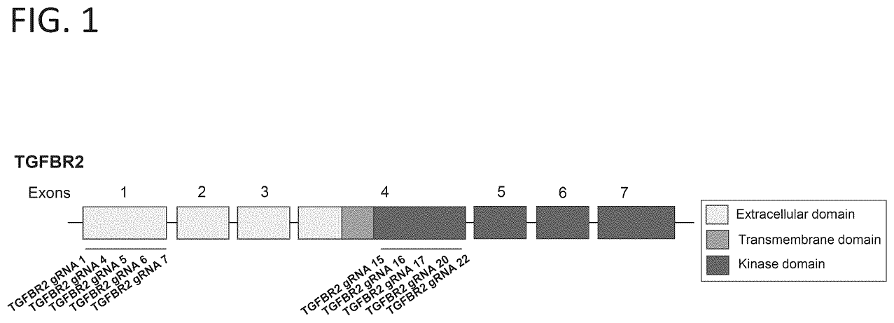

gRNAs that target a gene editing nuclease to various sites in human TGFBR2 exon 1 (extracellular domain of TGFBR2) or exon 4 (kinase domain of TGFBR2) were synthesized. FIG. 1 is a schematic showing the location of the nuclease cleavage sites directed by each gRNA. The sequences of exons 1 and 4 are presented in Table 1 below. The exon sequences are extracted from Ensembl canonical transcript ENS T00000295754.10. The target sequences of the gRNAs are presented in Table 2 below, where TGFBR2 gRNA target sequences are shown (5′-3′) with predicted cut site depicted (I) and PAM site in bold. Table 2 further indicates whether the target is on the sense (+) or antisense (−) strand of the TGFBR2 gene.

TABLE 1
TGFBR2 target exon sequences

| TGFBR2 | |
|---|---|
| Target Exon | Sequence (5′-3′) |
| Exon 1 | ACTCGCGCGCACGGAGCGACGACACCCCCGCGCGTGCACCCGCTCGGGACAG |
| (SEQ ID NO: 1) | GAGCCGGACTCCTGTGCAGCTTCCCTCGGCCGCCGGGGGCCTCCCCGCGCCT |
| | CGCCGGCCTCCAGGCCCCCTCCTGGCTGGCGAGCGGGCGCCACATCTGGCCC |
| | GCACATCTGCGCTGCCGGCCCGGCGCGGGGTCCGGAGAGGGCGCGGCGCGGA |
| | GGCGCAGCCAGGGGTCCGGGAAGGCGCCGTCCGCTGCGCTGGGGGCTCGGTC |
| | TATGACGAGCAGCGGGGTCTGCCATGGGTCGGGGGCTGCTCAGGGGCCTGTG |
| | GCCGCTGCACATCGTCCTGTGGACGCGTATCGCCAGCACGATCCCACCGCAC |
| | GTTCAGAAGTCGG |
| Exon 4 | AATATAACACCAGCAATCCTGACTTGTTGCTAGTCATATTTCAAGTGACAGG |
| (SEQ ID NO: 2) | CATCAGCCTCCTGCCACCACTGGGAGTTGCCATATCTGTCATCATCATCTTC |
| | TACTGCTACCGCGTTAACCGGCAGCAGAAGCTGAGTTCAACCTGGGAAACCG |
| | GCAAGACGCGGAAGCTCATGGAGTTCAGCGAGCACTGTGCCATCATCCTGGA |
| | AGATGACCGCTCTGACATCAGCTCCACGTGTGCCAACAACATCAACCACAAC |
| | ACAGAGCTGCTGCCCATTGAGCTGGACACCCTGGTGGGGAAAGGTCGCTTTG |
| | CTGAGGTCTATAAGGCCAAGCTGAAGCAGAACACTTCAGAGCAGTTTGAGAC |
| | AGTGGCAGTCAAGATCTTTCCCTATGAGGAGTATGCCTCTTGGAAGACAGAG |
| | AAGGACATCTTCTCAGACATCAATCTGAAGCATGAGAACATACTCCAGTTCC |
| | TGACGGCTGAGGAGCGGAAGACGGAGTTGGGGAAACAATACTGGCTGATCAC |
| | CGCCTTCCACGCCAAGGGCAACCTACAGGAGTACCTGACGCGGCATGTCATC |
| | AGCTGGGAGGACCTGCGCAAGCTGGGCAGCTCCCTCGCCCGGGGGATTGCTC |
| | ACCTCCACAGTGATCACACTCCATGTGGGAGGCCCAAGATGCCCATCGTGCA |
| | CAGGGACCTCAAGAGCTCCAATATCCTCGTGAAGAACGACCTAACCTGCTGC |
| | CTGTGTGACTTTGGGCTTTCCCTGCGTCTGGACCCTACTCTGTCTGTGGATG |
| | ACCTGGCTAACAGTGGGCAG |

TABLE 2
TGFBR2 gRNA target sequences

| | | | Sense (+) | | |
|---|---|---|---|---|---|
| | | TGFBR2 | or anti- | TGFBR2 | SEQ |
| TGFBR2 gRNA | | Exon | sense (−) | Domain | ID |
| ID | Target Sequence (5′-3′) | Targeting | strand | Targeting | NO: |
| TGFBR2 gRNA 1 | TGCTGGCGATACGCGTC\|CACAGG | 1 | − | ECD | 3 |
| TGFBR2 gRNA 4 | TCGGTCTATGACGAGCA\|GCGGGG | 1 | + | ECD | 4 |
| TGFBR2 gRNA 5 | AACGTGCGGTGGGATCG\|TGCTGG | 1 | − | ECD | 5 |
| TGFBR2 gRNA 6 | GGACGATGTGCAGCGGC\|CACAGG | 1 | − | ECD | 6 |
| TGFBR2 gRNA 7 | CTCGGTCTATGACGAGC\|AGCGGG | 1 | + | ECD | 7 |
| TGFBR2 gRNA 15 | CAAGAGGCATACTCCTC\|ATAGGG | 4 | − | ICD | 8 |
| TGFBR2 gRNA 16 | CCACGCCAAGGGCAACC\|TACAGG | 4 | + | ICD | 9 |
| TGFBR2 gRNA 17 | CCAAGATGCCCATCGTG\|CACAGG | 4 | + | ICD | 10 |
| TGFBR2 gRNA 20 | AAAGCGACCTTTCCCCA\|CCAGGG | 4 | − | ICD | 11 |
| TGFBR2 gRNA 22 | GCCGCGTCAGGTACTCC\|TGTAGG | 4 | − | ICD | 12 |

Healthy donor human T cells were activated with anti-CD3/CD28 beads Thermo Fisher, #40203D at a 3:1 ratio (beads:CD3⁺ cells) for 48 hours before being electroporated with 1 μM TGFBR2 targeting RNPs (CRISPR-Cas9 gRNA ribonucleoprotein) using the Lonza 4D nucleofector system, program EH-115. Simultaneously, the endogenous TCR was knocked out and the expression of an exogenous TCR (1G4) was induced. Following expansion of the T cells in AIM-V media Thermo Fisher, #A3830801 containing 5% human serum Sigma-Aldrich, #H4522, 1% glutamax Thermo Fisher, #35050061, 5 μg/mL gentamicin Thermo Fisher, #15750037, IL-7 (5 ng/mL) Peprotech, #200-07, and IL-15 (5 ng/mL) Peprotech, #200-15 for 7 days, the T cells were treated with or without TGF-β (20 ng/mL) R&D systems, #240-B-010/CF for 30 minutes, before being intracellularly stained with an antibody detecting phosphorylated SMAD2/3 protein BD Bioscience, #562696.

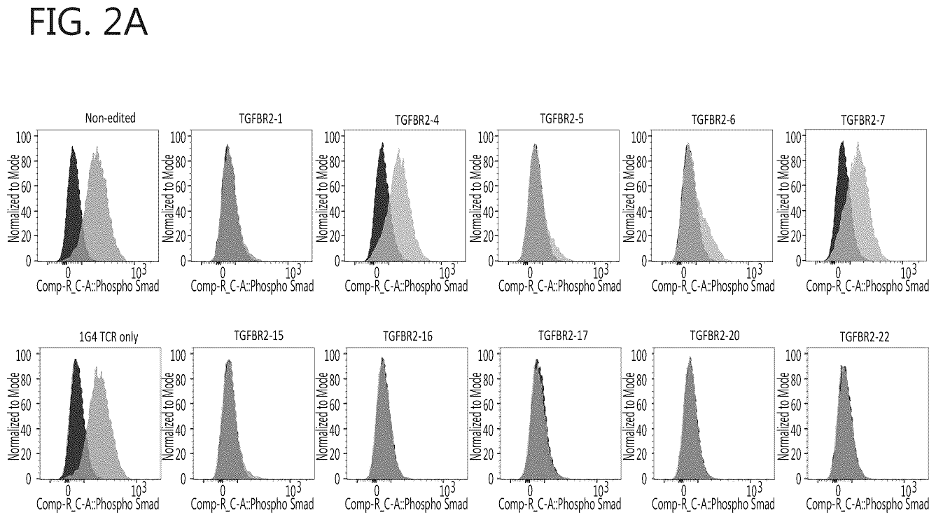

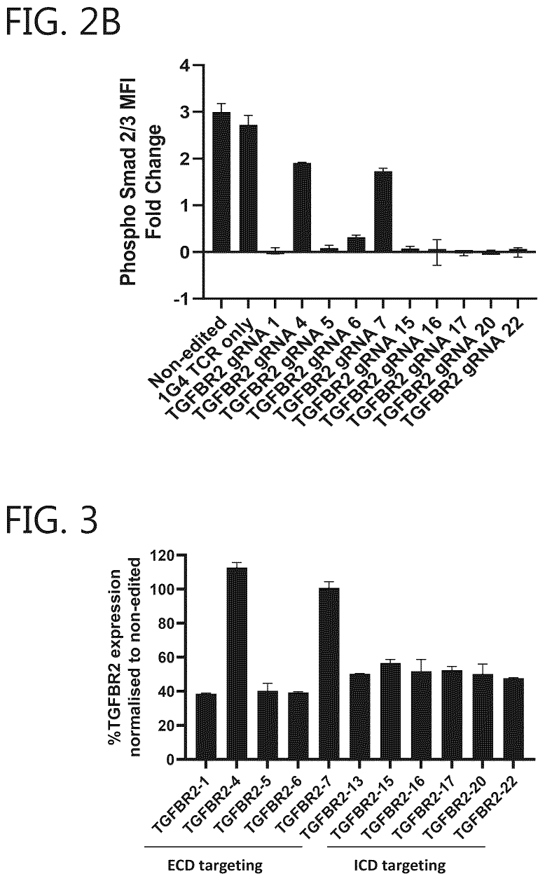

As shown in FIGS. 2A and 2B, T cells lacking both TGFBR2 editing and 1G4 TCR expression (“non-edited”) and T cells lacking TGFBR2 editing but expressing exogenous 1G4 TCR (“1G4 only”) displayed an increase in phosphorylated SMAD2/3 following TGF-β treatment, whereas many of the TGFBR2-disrupted, 1G4 TCR-expressing T cells did not demonstrate any increase. Some of the TGFBR2 exon 1-edited, 1G4 TCR-expressing T cells, and all of the TGFBR2 exon4-edited, 1G4 TCR-expressing T cells, were completely resistant to TGF-β signaling.

As shown in FIG. 3, gRNAs 4 and 7 guided edits to the TGFBR2 gene that were inefficient at reducing TGFBR2 surface expression on 1G4 TCR-expressing T cells. All other TGFBR2 gRNAs tested resulted in reduced TGFBR2 surface expression, including all gRNAs targeting exon 4 (FIG. 3). Although the TGFBR2 gRNAs targeting the intracellular kinase domain (exon 4) all reduced surface expression of TGFBR2 as compared to control cells, the exon 4-edited T cells showed a trend of greater TGFBR2 surface expression as compared to the exon 1 (extracellular domain)-edited cells in which surface expression was successfully reduced (gRNAs TGFBR2-1, TGFBR2-5, and TGFBR2-6). Surface expression data are presented in Table 3 below.

TABLE 3
TGFBR2 median fluorescence intensity
(MFI) of TGFBR2 edited T cells

| | TGFBR2 MFI | | |
|---|---|---|---|
| Sample | Replicate 1 | Replicate 2 | Average |
| 1G4 TCR only | 606 | 671 | 638.5 |
| TGFBR2-1 | 248 | 244 | 246 |
| TGFBR2-4 | 733 | 707 | 720 |
| TGFBR2-5 | 277 | 237 | 257 |
| TGFBR2-6 | 253 | 250 | 251.5 |
| TGFBR2-7 | 628 | 660 | 644 |
| TGFBR2-13 | 320 | 322 | 321 |
| TGFBR2-15 | 352 | 371 | 361.5 |
| TGFBR2-16 | 299 | 362 | 330.5 |
| TGFBR2-17 | 345 | 325 | 335 |
| TGFBR2-20 | 347 | 293 | 320 |
| TGFBR2-22 | 302 | 306 | 304 |

### 6.6.2. Example 2: TGFBR2-Disrupted, TCR-Expressing, T Cells Exhibit Superior Functionality in the Presence of TGF-β

This example shows the ability of TGFBR2-disrupted T cells to maintain cytotoxic activity through multiple rounds of antigen exposure.

1G4 TCR-expressing, TGFBR2-disrupted T cells were generated from healthy human donors as in Example 1. Following 14 days of T cell expansion in AIM-V media Thermo Fisher, #A3830801 containing 5% human serum Sigma-Aldrich, #H4522, 1% glutamax Thermo Fisher, #35050061, 5 μg/mL gentamicin Thermo Fisher, #15750037, IL-7 (5 ng/mL) Peprotech, #200-07, and IL-15 (5 ng/mL) Peprotech, #200-15, T cells were subjected to a repetitive cytotoxicity assay using the IncuCyte platform.

T cells were co-cultured with GFP-expressing A375 cells, which express the 1G4 TCR cognate antigen, the peptide HLA (pHLA) complex of NY-ESO-1 and HLA-A*02:01, at an effector-to-target ratio of 5:1, in the presence or absence of exogenous TGF-β (20 ng/mL) R&D systems, #240-B-010/CF for approximately 72 hours. T cells were then harvested and co-cultured with fresh A375-GFP⁺ cells for a total of 4 rounds of tumor challenge. Images were obtained using a 10× objective every 2 hours and T cell cytotoxicity was determined by measuring the number of GFP⁺ A375 cells remaining in the co-cultures.

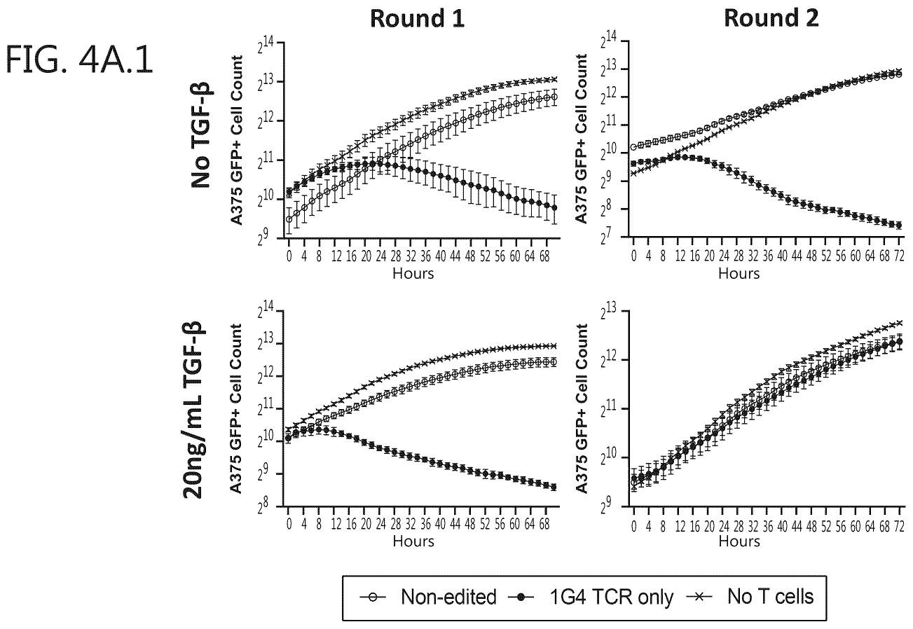

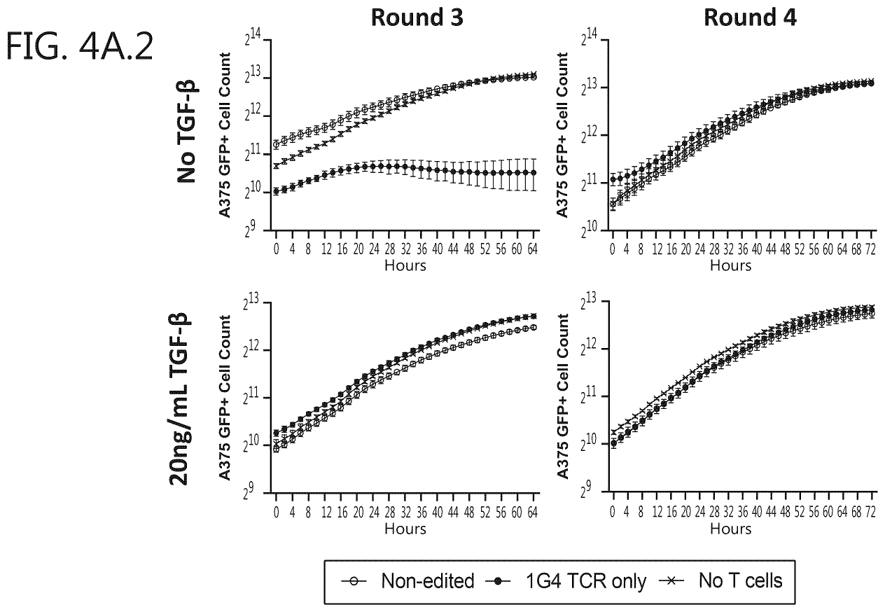

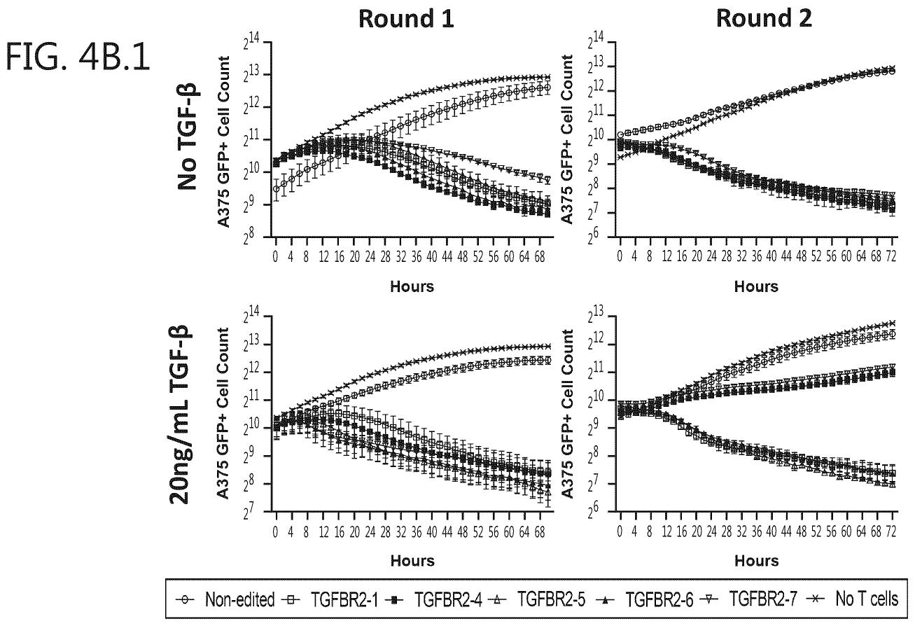

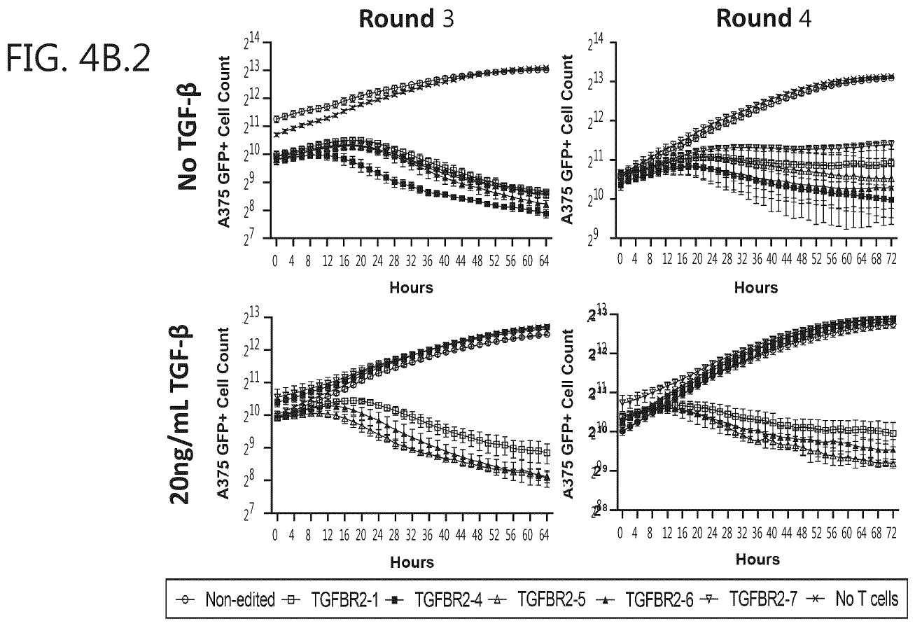

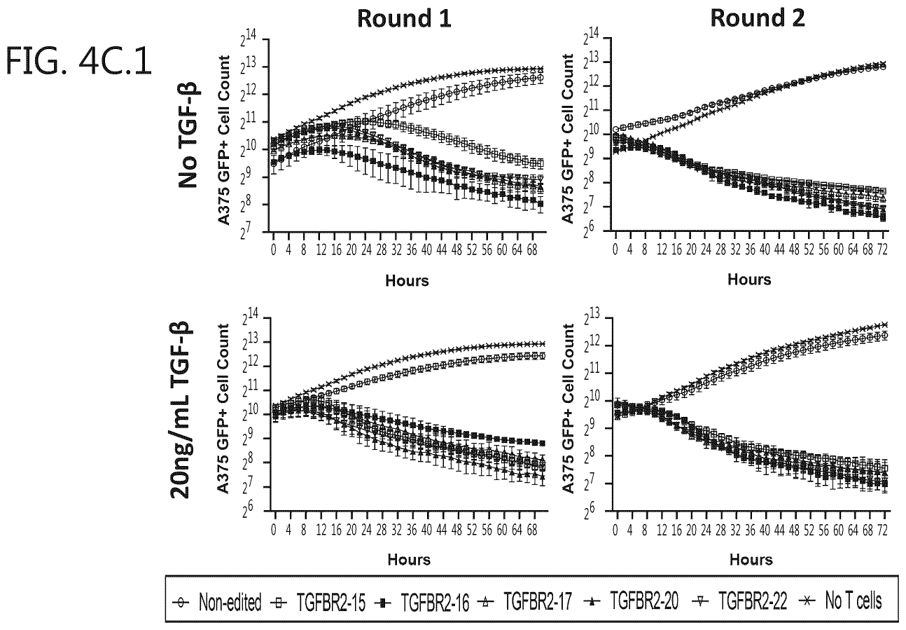

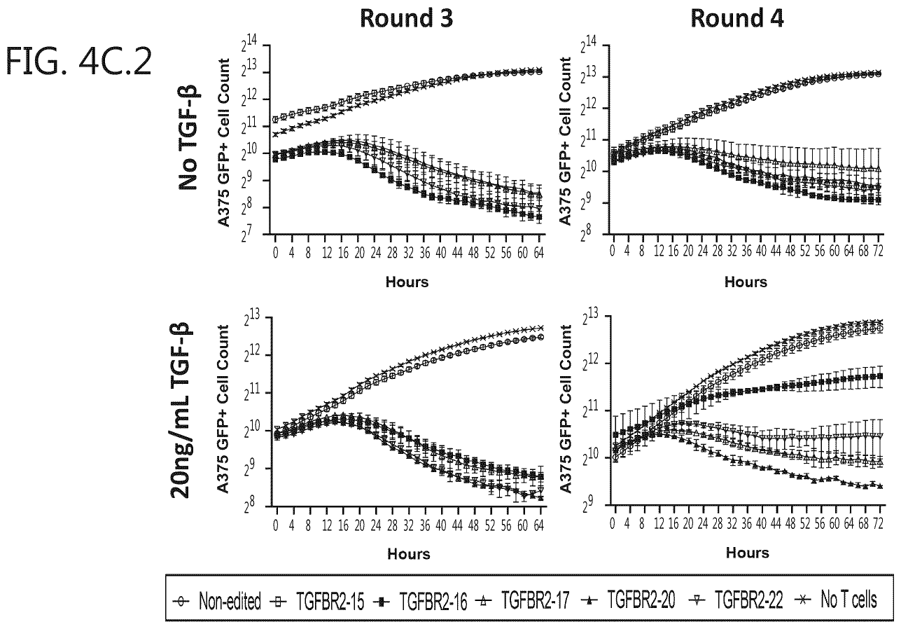

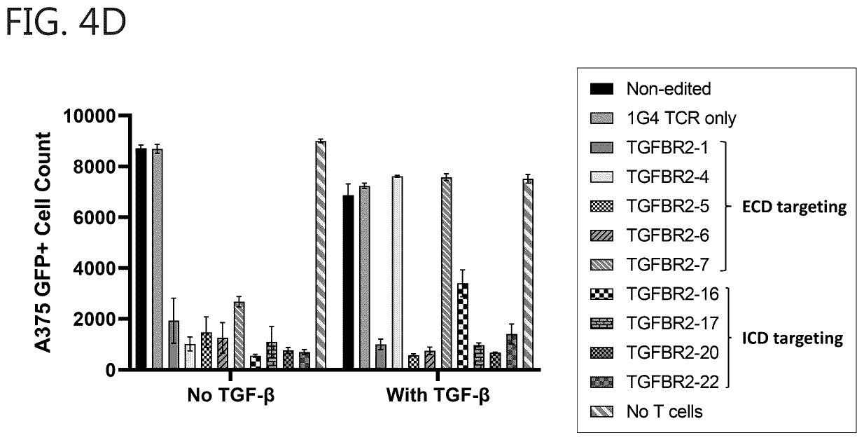

During round 1, all 1G4 TCR-expressing T cells were able to control A375-GFP⁺ cell growth in the presence or absence of TGF-β (FIGS. 4A.1, 4B.1, 4C.1).

During round 2, non-TGFBR2 disrupted, 1G4-expressing T cells (“1G4 TCR only”) could no longer control the growth of A375-GFP⁺ cells in the presence of TGF-β (FIG. 4A.1). TGFBR2-disrupted T cells with reduced TGFBR2 expression and reduced TGF-β signaling ability continued to be able to kill A375-GFP⁺ cells, whether in the presence of TGF-β or not, during the second and subsequent rounds of tumor cell challenge (FIGS. 4B.1 and 4B.2, exon 1 disruptions; FIGS. 4C.1 and 4C.2, exon 4 disruptions). TGFBR2 gRNA 4-edited and TGFBR2 gRNA 7-edited T cells, which still retained some functional TGF-β signaling (FIG. 2B), had reduced ability to control A375-GFP⁺ cell growth in the presence of TGF-β beginning in the second round of killing (FIG. 4B.1).

In the absence of exogenous TGF-β, 1G4 TCR only T cells lost their cytotoxic function during rounds 3 and 4 of tumor cell challenge, while TGFBR2 disrupted T cells continued to control the growth of A375-GFP⁺ cells in the absence of TGF-β (FIGS. 4A.2, 4B.2 and 4C.2). The final round 4 A375-GFP⁺ cell counts are shown in FIG. 4D.

These data show that TGFBR2-disrupted T cells are resistant to TGF-β-mediated suppression of cytotoxic function and are more potent at killing antigen-positive tumor cells in the presence or absence of exogenous TGF-β.

### 6.6.3. Example 3: TGFBR2-Disrupted, TCR-Expressing, T Cells Maintain Proliferative Capacity in the Presence of TGF-β

This example shows that TGFBR2-disrupted T cells are capable of continued proliferation in the presence of TGF-β.

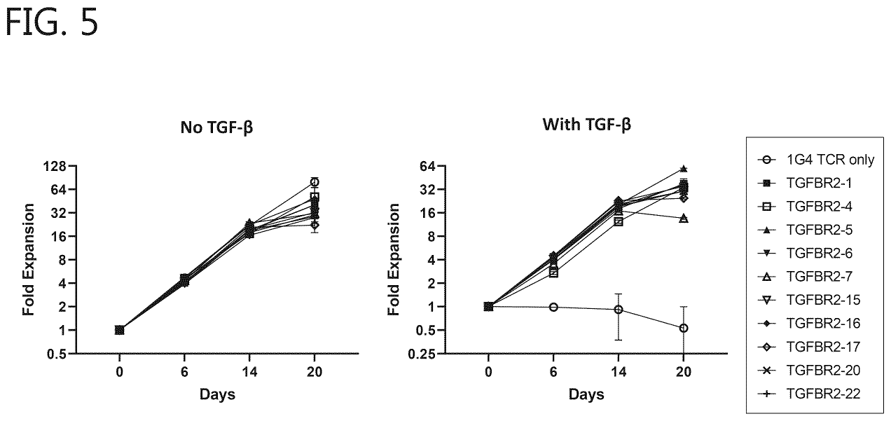

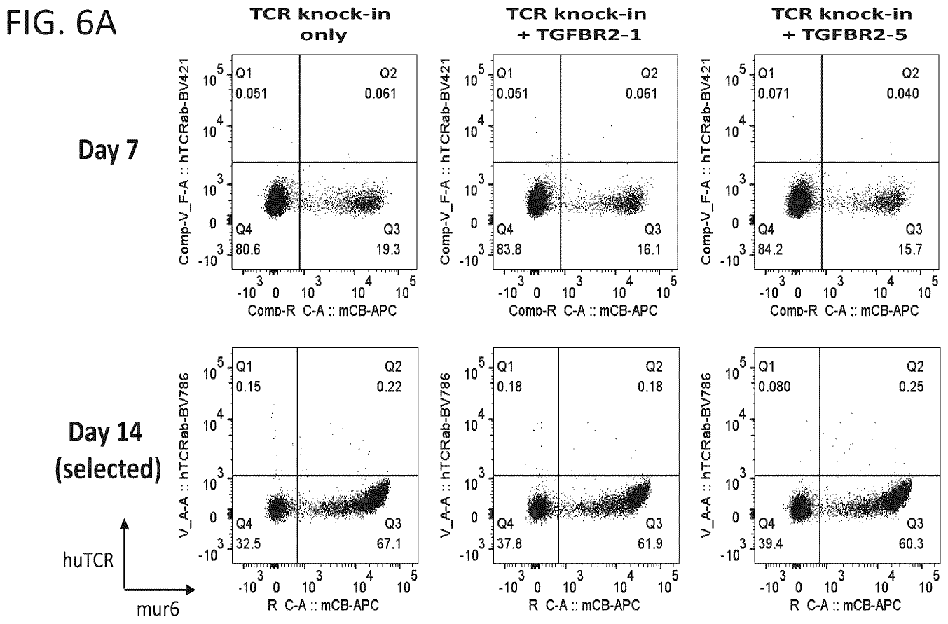

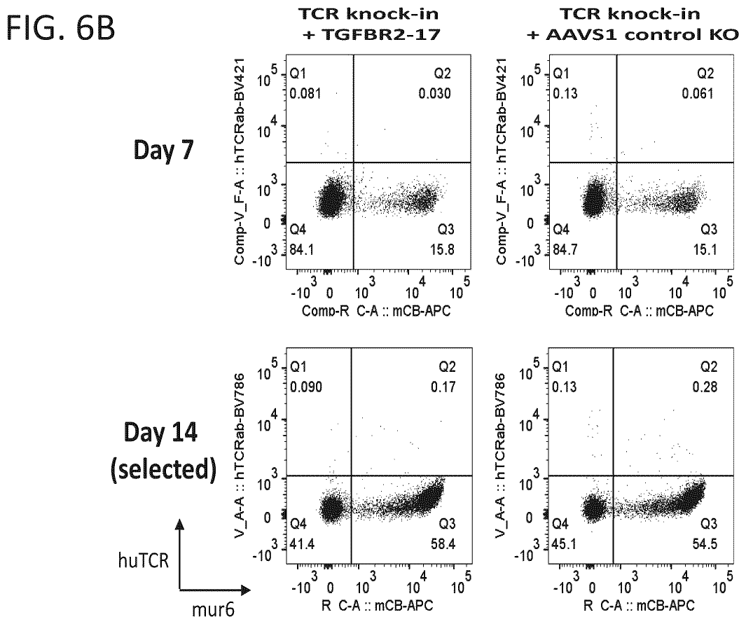

1G4 TCR-expressing, TGFBR2-disrupted, T cells were subjected to repetitive restimulation using ImmunoCult (anti-CD3/CD28/CD2) Stem Cell Technologies, #10990, in the presence or absence of TGF-β (20 ng/mL) R&D systems, #240-B-010/CF. T cell proliferation was quantified by counting T cells on a weekly basis (FIG. 5). While 1G4 TCR-only T cells were unable to expand in the presence of TGF-β, TGFBR2-disrupted T cells maintained their ability to proliferate to the same degree as in the absence of TGF-β. TGFBR2 gRNA 4-edited and TGFBR2 gRNA-7 edited T cells, which still retained some functional TGF-β signaling (FIG. 2B), expanded more slowly in the presence of TGF-β, compared to other TGFBR2-disrupted T cells.

This data shows that TGFBR2 KO T cells are resistant to TGF-β-mediated suppression of proliferation and can maintain their proliferative capacity in the presence of TGF-β.

### 6.6.4. Example 4: TGFBR2-Disrupted, TCR-Expressing, T Cells have Similar Levels of TCR Expression

This example shows that TGFBR2-disrupted T cells express similar levels of exogenous TCR as non-disrupted cells.

TCR knock-in, TGFBR2-disrupted T cells were engineered by co-electroporating TGFBR2-, TRAC- and TRBC-targeting Cas9-gRNA ribonucleoprotein complexes (RNPs) and a homology directed repair DNA template encoding a mutant DHFR gene that is resistant to methotrexate and an exogenous TCR containing a mur6 epitope in the Cβ domain (as described in U.S. patent application Ser. No. 17/557,514, which is incorporated herein in its entirety), with homology arms for insertion in the TRAC locus.

Cells were expanded for seven days after electroporation and then selected using methotrexate according to methods described in US Pat. Pub. No. 2022/0041999 (incorporated herein by reference in its entirety). Expression of endogenous TCR was determined by staining with an antibody recognizing the human TCRαβ complex (antibody clone IP26). Expression of the exogenous TCR was detected with an antibody that recognizes the mur6 epitope (H57).

Seven days after electroporation, T cells expressed the exogenous TCR at equivalent levels in all conditions tested (no TGFBR2 disruption, TGFBR2 disruption with three different targeting sequences, and an AAVS1 control KO) both before selection at seven days post-electroporation and after selection 14 days post-electroporation (FIG. 5)

This data shows that TGFBR2 KO T cells do not have impaired ability to express an exogenous TCR.

### 7. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. A T cell comprising an engineered genomic modification of the TGFBR2 gene, wherein the engineered genomic modification results in a level of surface-expressed TGFBR2, or a detectable portion thereof, that is between about 20% and about 60% of the level of surface-expressed TGFBR2 on a matched control cell.

2. The T cell of claim 1, wherein the T cell is a CD8+ αβ T cell, a CD4+ αβ T cell, or a γδ T cell.

3. The T cell according to claim 1, wherein the T cell is a human T cell.

4. The T cell according to claim 1, wherein the surface-expressed TGFBR2, or detectable portion thereof, is capable of binding TGF-β but not phosphorylating TGFBR1.

5. The T cell according to claim 1, wherein the T cell cannot effectively signal through Smad2/3 in response to contact of the T cell with physiologically relevant levels of TGF-β.

6. The T cell according to claim 1, wherein the engineered genomic modification comprises one or more of (i) an insertion and/or a deletion in the TGFBR2 gene promoter, (ii) a frame-shifting insertion and/or deletion in an exon of the TGFBR2 gene, (iii) a deletion of a part, but not the entirety, of the coding region of the TGFBR2 gene, (iv) a substitution, insertion, and/or deletion that creates a stop codon in an exon upstream of the native stop codon, and (v) a substitution, insertion, and/or deletion that modifies one or more donor and/or acceptor sites RNA splice sites within the TGFBR2 gene.

7. The T cell according to claim 1, wherein the genomic modification is in exon 4 of the TGFBR2 gene.

8. The T cell of claim 7, wherein the genomic modification is a frameshift caused by an RNA-guided nuclease cut between bases 294 and 295, 389 and 390, 543 and 544, 547 and 548, or 674 and 675 of exon 4 of the TGFBR2 gene (SEQ ID NO: 2).

9. The T cell of claim 1, wherein the T cell expresses an exogenous TCR or a CAR, optionally an exogenous TCR.

10. The T cell of claim 9, wherein the T cell expresses an exogenous TCR.

11. The T cell of claim 10, wherein the exogenous TCR recognizes a tumor antigen, optionally a tumor neoantigen.

12. The T cell of claim 11, wherein the tumor antigen is a neoantigen.

13. The T cell of claim 12, wherein the tumor antigen is a shared tumor neoantigen.

14. The T cell of claim 12, wherein the tumor antigen is a non-shared tumor neoantigen.

15. The T cell of claim 9, wherein the T cell maintains the ability to kill a population of target cells that express the antigen recognized by the exogenous TCR or CAR in vitro in the presence of physiologically relevant levels of TGF-β after at least two exposure events to the target cells.

16. The T cell of claim 9, wherein the T cell maintains the ability to kill a population of target cells that express the antigen recognized by the exogenous TCR or CAR in vitro for at least about 72 hours in the presence of physiologically relevant levels of TGF-β.

17. A T cell comprising an engineered genomic modification of the TGFBR2 gene, wherein the modification results in a surface-expressed TGFBR2 that is truncated.

18-31. (canceled)

32. A pharmaceutical composition comprising a T cell having an engineered genomic modification of the TGFBR2 gene, wherein the engineered genomic modification results in a level of surface-expressed TGFBR2, or a detectable portion thereof, that is between about 20% and about 60% of the level of surface-expressed TGFBR2 on a matched control cell and a pharmaceutically acceptable carrier.

33-34. (canceled)

35. A method of engineering a T cell, comprising modifying the TGFBR2 gene in the T cell genome, wherein following gene modification the level of surface-expressed TGFBR2 or a detectable portion thereof is between about 20% and about 60% of the level of surface-expressed TGFBR2 on a matched control cell.

36-53. (canceled)

54. A method of engineering a T cell, comprising modifying the TGFBR2 gene in the T cell genome, wherein the modification is within exon 4 and results in a surface-expressed TGFBR2 that is truncated.

55-73. (canceled)

74. An engineered T cell produced by a method comprising modifying the TGFBR2 gene in the T cell genome, wherein following gene modification the level of surface-expressed TGFBR2 or a detectable portion thereof is between about 20% and about 60% of the level of surface-expressed TGFBR2 on a matched control cell.

75. A pharmaceutical composition comprising a engineered T cells produced by a method comprising modifying the TGFBR2 gene in the T cell genome, wherein following gene modification the level of surface-expressed TGFBR2 or a detectable portion thereof is between about 20% and about 60% of the level of surface-expressed TGFBR2 on a matched control cell, and a pharmaceutically acceptable carrier.

76. A method of treating a patient, comprising:
administering to the patient a therapeutically effective amount of T cells having an engineered genomic modification of the TGFBR2 gene, wherein the engineered genomic modification results in a level of surface-expressed TGFBR2, or a detectable portion thereof, that is between about 20% and about 60% of the level of surface-expressed TGFBR2 on a matched control cell.

77-79. (canceled)

80. A pharmaceutical composition comprising T cells having an engineered genomic modification of the TGFBR2 gene, wherein the engineered genomic modification results in a level of surface-expressed TGFBR2, or a detectable portion thereof, that is between about 20% and about 60% of the level of surface-expressed TGFBR2 on a matched control cell and a pharmaceutically acceptable carrier.

81. The pharmaceutical composition of claim 80, wherein the composition is adapted for administration by intravenous infusion.

82. The pharmaceutical composition of claim 80, wherein the composition is adapted for intratumoral administration.

83. The pharmaceutical composition of claim 80, wherein the exogenous TCR or CAR is integrated into a defined place in the genome of the T cell.

84. The method of claim 83, wherein the integration is performed using CRISPR, optionally CRISPR-Cas9.

85. A pharmaceutical composition comprising T cells having an engineered genomic modification of the TGFBR2 gene, wherein the modification results in a surface-expressed TGFBR2 that is truncated.
